(19) United States
(12) Patent Application Publication
(10) Pub. No.: US 2023/0310330 A1
(43) Pub. Date: Oct. 5, 2023

(54) PEPTIDIC OLIGOMERS AND METHODS OF USING THE SAME

(71) Applicants: BIOVENTURES, LLC, Little Rock, AR (US); THE BOARD OF TRUSTEES OF THE UNIVERSITY OF ARKANSAS, Little Rock, AR (US)

(72) Inventors: Zeeshan Habeeb, Pine Bluff, AR (US); Ruud Dings, Little Rock, AR (US)

(21) Appl. No.: 18/131,280

(22) Filed: Apr. 5, 2023

Related U.S. Application Data
(60) Provisional application No. 63/327,643, filed on Apr. 5, 2022.

(51) Int. Cl. A61K 9/50; A61K 31/26; A61P 35/00

(52) U.S. Cl. CPC A61K 9/5052; A61K 31/26; A61P 35/00

(57) ABSTRACT

Disclosed herein are biocompatible peptidic oligomers and methods of using the same where the biocompatible peptidic oligomer encapsulates an agent and the biocompatible peptidic oligomer comprises a linear chain formed from a multiplicity of independently selected nonproteogenic amino acids joined by amide bonds and the nonproteogenic amino acids comprise two or more optionally substituted methylene groups interposed between a terminal amino and a terminal carboxylic acid or ester group.

### CROSS-REFERENCE TO RELATED APPLICATIONS

This application claims benefit of priority to U.S. Patent Application No. 63/327,643, filed Apr. 5, 2022, the contents of which is incorporated by reference in its entirety.

### STATEMENT REGARDING FEDERALLY SPONSORED RESEARCH

This invention was made with government support under P20 GM103429 awarded by the National Institutes of Health (NIH). The government has certain rights in the invention.

### FIELD OF THE INVENTION

The disclosed technology is generally directed to biocompatible peptidic oligomers encapsulating agents. More particularly the technology is directed to encapsulating small molecules in biocompatible peptidic oligomers for improved solubility and bioavailability.

### BACKGROUND OF THE INVENTION

While many drug delivery vehicles have shown promise at the research stage, they fail at clinical implementation because there is no control over the size of the resulting nanostructures self-assembly process. This can lead to liver and kidney damage when excreted from the body. As a result, there is a need for better drug delivery vehicles.

### BRIEF SUMMARY OF THE INVENTION

Disclosed herein are peptidic oligomers and methods of using the same. One aspect of the technology provides for a complex comprising a peptidic oligomer encapsulating an agent, or a pharmaceutically acceptable salt thereof, wherein the peptidic oligomer comprises a linear chain formed from a multiplicity of independently selected nonproteogenic amino acids joined by amide bonds and the nonproteogenic amino acids comprise two or more optionally substituted methylene groups interposed between a terminal amino and a terminal carboxylic acid or ester group. In some embodiments, the nonproteogenic amino acids comprise optionally substituted β-amino acids, such as β-alanine. In other embodiments, the nonproteogenic amino acids comprise optionally substituted γ-amino acids, such as γ-alanine. In some embodiments, the peptidic oligomer is substituted with one or more targeting moieties, steric groups, saccharide moieties, labels, or any combination thereof. In some embodiments, the peptidic oligomer comprises between 4-8 nonproteogenic amino acids. In some embodiments, 1 peptidic oligomer encapsulates 1 agent. In some embodiments, the agent may be an active pharmaceutical ingredient, a diagnostic agent, or an agrochemical.

Another aspect of the technology provides for pharmaceutical compositions comprising an active pharmaceutical ingredient encapsulated by any of the peptidic oligomers described herein and a pharmaceutically acceptable carrier, excipient, or diluent.

Another aspect of the technology provides for a method for treating a subject in need of an active pharmaceutical ingredient comprising administering any of the pharmaceutical compositions described herein to the subject.

Another aspect of the technology provides for a method for treating a subject in need of a treatment for cancer comprising administering a pharmaceutical composition comprising a peptidic oligomer and a pharmaceutically acceptable carrier, excipient, or diluent to the subject. The peptidic oligomer may comprise a linear chain formed from a multiplicity of independently selected nonproteogenic amino acids joined by amide bonds. The nonproteogenic amino acids comprise two or more optionally substituted methylene groups interposed between a terminal amino and a terminal carboxylic acid or ester group.

Another aspect of the technology provides for a method for preparing the composition. The method may comprise incubating the agent with the peptidic oligomer in a solvent under conditions sufficient for encapsulating the agent with the peptidic oligomer.

These and other aspects of the technology will be further described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: An Avogadro model predicting the diameter (Angstroms) of a six-alanine chain alpha helix. The diameter increases as the α alanine expanded to β and γ.

FIG. 2: An Avogadro model predicting the hydrogen bonding distance (Angstroms) of a six-alanine chain alpha helix. Alanine's alpha helix was widened and the distance, in Angstroms, between the hydrogen bonds decreased indicating that the molecules increase in strength as the chain expands.

FIG. 3: Circular dichroism spectra of the α, β, γ-chains in trifluoroethanol. Trifluoroethanol is a strong protic solvent that promotes hydrogen bonding and is known to stabilize the alpha helix of proteins. Peaks at ˜193, 208, and 222 nm indicate the ability to form alpha helices.

FIG. 4: Toxicity assay of the empty beta and gamma chains performed with mouse 4T1 breast cancer cells. Cisplatin was used as a toxicity reference. Cells were incubated with varying concentrations for 48 hours, followed by CCK8 reagent. The absorbance at 450 nm was recorded. This data indicates that as the concentration of cisplatin increases, the number of viable cells decreases. As predicted, empty beta and gamma alpha helices show biocompatibility in that they do not display cellular toxicity.

FIG. 5: A schematic of proposed expanded α-helix system and the skeletal structures of alanine, beta, and gamma.

FIG. 6: A skeletal structure of Sulforaphane, an extract of broccoli, that has demonstrated anti-cancer properties with 4T1 breast cancer cells. It is an oil with poor bioavailability.

FIG. 7: Encapsulation efficiency for B6. Saturated solutions of B6 in water and sulforaphane in ethanol, at various molar ratios, were incubated overnight at room temperature to allow encapsulation. The unprocessed solutions were analyzed by mass spectrometry to determine encapsulation efficiency (EE). B6 shows 90% encapsulation efficiency at 1:0.5 molar ratio.

FIG. 8: Encapsulation efficiency for G6. Saturated solutions of G6 in water and sulforaphane in ethanol, at various molar ratios, were incubated overnight at room temperature to allow encapsulation. The unprocessed solutions were analyzed by mass spectrometry to determine encapsulation efficiency (EE). G6 has a maximum EE of 60% at 1:1.25 molar ratio.

FIG. 9: In vitro assay for cell viability versus concentration of B6, G6, free sulforaphane, sulforaphane encapsulated by B6 (called “BS”), and sulforaphane encapsulated by G6 (called “GS”) were tested for biological activity against 4T1 breast cancer cells. The assay was run for 72 hours with the CellTiter-Glo assay to determine cell viability. Results show that the free peptides B6 and G6 are biocompatible and do not affect cell viability. Free sulforaphane shows significant activity at 10 mM. The encapsulated constructs, BS and GS, show significant activity at as low as 0.1 mM, 100× less than the free sulforaphane.

FIG. 10: Pharmaceutical absorption (Absorption, distribution, metabolism, and excretion; ADME) rates. Using Schrodinger, Qikprop acts as a rapid AADME prediction of drug candidates. Hexa alanine, Beta, Gamma, and sulforaphane were analyzed for relevant pharmaceutically relevant properties. Alanine exhibits properties that fall outside of normal range predictions to perform well in clinical testing. Hexa, Beta, and Gamma shows greater probability of success (Qppolrz) in living organisms than sulforaphane. Sulforaphane indicated the least biocompatibility within kidney (QPPMDCk), binding to albumin (QPlogKhsa), and a weak polar component (WPSA), as expected for an oil.

FIG. 11: Structures of B6 (M/W 1135 g/mol; above) and G6 (M/W 1219 g/mol; below) with Boc groups intact as bulky side groups that are chemically inert.

FIG. 12: H-bond distances in Angstroms. H-bond distances in Angstroms calculated for hexa peptides of alanine (A6), lysine (K6), arginine (R6), B6 and G6 orientated into an alpha helical structure, using Avogadro and relaxed with a MMFF94 forcefield.

FIG. 13: Electrostatic potential maps of R6, B6 and G6 left to right looking down C-terminal end of peptide.

FIG. 14: B6-FITC uptake is not by direct translocation. (A) Untreated at 4° C. B6-FITC treated at (B) 4° C. and (C and D) 37° C. Panel C FITC channel only. Blue=Hoechst 33342 nuclear stain; Green=B6-FITC. Magnification 20×; Scale bar=20.

FIG. 15: Bafilomycin Al blocks CPP translocation. (A) FITC-labeled B6 and G6 are easily detected by flow cytometry. (B) BafA1 blocks B6-FITC binding and translocation as noted by the reduced FITC signal.

FIG. 16: (A and B) B6Sulf did not induce any decrease in individual body weights, an indirect measure of general toxicity. Total amount of (C and D) leukocytes, (E and F) erythrocytes, and (G and H) thrombocytes in the circulation are not affected by B6Sulf treatment. B6Sulf 2 mg/mg i.p.; Vehicle=H2O. Lines graphs show 2 hrs and 24 hrs after injection per individual. Bar graphs show means and S.D. WBC—white blood cell count; RBC=red blood cell count; PLT=platelets

FIG. 17: B6Sulf did not induce any significant decrease in (A and B) lymphocytes, (C and D) monocytes, or (E and F) neutrophils as measured in total amount or percentage of leukocyte composition. B6Sulf 2 mg/mg i.p.; Vehicle=H2O. Lines graphs show 2 hrs and 24 hrs after injection per individual. LYM=lymphocytes; MON=Monocytes; NEU=Neutrophils

FIG. 18: B6Sulf did not induce any significant changes in (A) hemoglobin, (B) hematocrit, (C) mean corpuscular volume, (D) mean corpuscular volume hemoglobin, (E) red cell distribution width or (D) percentage. B6Sulf 2 mg/mg i.p.; Vehicle=H2O. Lines graphs show 2 hrs and 24 hrs after injection per individual. HGB=hemoglobin; HCT=hematocrit; MCV=mean corpuscular volume; MCHC=mean corpuscular hemoglobin; RDWc=red cell distribution width; fl=femtoliters

FIG. 19: B6Sulf did not induce any significant changes in (A) the percentage of platelets in blood, (B) volume of platelets, (C) platelet distribution width or (D) percentage. B6Sulf 2 mg/mg i.p.; Vehicle=H2O. Lines graphs show 2 hrs and 24 hrs after injection per individual. PCT=plateletcrit; MPV=mean platelet volume; PDW=platelet distribution width; fl=femtoliters

FIG. 20: (A), Tumor growth curves are shown in mean volumes±SEM with nonlinear regression-fit lines, and (B) tumor weights are shown as individual values and the means of tumor masses±SEM (n=4-8 animals per group pooled from two individual experiments). Treatments were initiated on day 5 (indicated by arrow) when tumor were established and an average size of 7 mm in diameter (˜150 mm³). The vehicle and compounds were injected daily intraperitoneal in 150 ul volumes. B6 and sulforaphane were injected in the molar equivalent doses as the quantities found in 2 mg/kg B6Sulforaphane group. *, P<0.05, **, P<0.01, ***, P<0.05, two-sided t test; ns, not significant.

FIG. 21: B6Sulforophane treatment does not induce general toxicity in 4T1-bearing mice. No significant changes in body weights were observed. Treatments were initiated on day 5. Data are shown as the means of body weight in g±SD (n=4-8 animals per group pooled from 2 individual experiments).

FIG. 22: B6Sulforophane treatment does not induce general splenocytes toxicity in 4T1-bearing mice. Data are shown as individual values and the means of spleen weights in g±SD (n=4-8 animals per group pooled from 2 individual experiments). ns, not significant as determined by two-sided t test.

FIG. 23: The small intestines (A), ceca (B), and colons (C) of 4T1-bearing mice. Data are shown as individual values and the means of small intestine or colon lengths in cm±SD or weights in g±SD for ceca (n=4-8 animals per group pooled from 2 individual experiments). ns, not significant as determined by two-sided t test.

### DETAILED DESCRIPTION OF THE INVENTION

Disclosed herein are peptidic oligomers and methods of using the same. The peptidic oligomers described herein allow for a single molecule self-assembles into an a well characterized alpha-helical structure. The peptidic oligomers may be biocompatible and are capable of encapsulating individual small molecules of therapeutic interest, resulting in the improvement of their solubility and bioavailability, thus reducing dosage and the likelihood of side effects. The peptidic oligomers may also provide therapeutic benefit without an encapsulated molecule or sensitize a subject to an encapsulated molecule. Additionally, the peptidic oligomers described herein may be cell penetrating peptides (CPPs) that can enter a cell. Since the oligomers may be synthesized using a solid resin support, there is complete control over the resulting length. By having complete control over the length of the peptidic oligomers and how much of the small molecule is encapsulated, the drug delivery vehicles are uniformly small allowing for renal and hepatic clearance, and the concentration of the encapsulant is known.

Peptidic oligomers refers to oligomers forming a linear chain of amide linked amino acids. As used herein, oligomer refers to compounds comprising 4 to 20 amino acids, e.g., 4, 5, 6, 7, 8, 9, 10, 11, 12, 13, 14, 15, 16, 17, 18, 19, or 20 amino acids. In some embodiments, oligomers comprise 4 to 10 amino acids or 4 to 8 amino acids.

The peptidic oligomers comprise a multiplicity of nonproteogenic amino acids. In some embodiments, all of the amino acids in the peptidic oligomer comprise nonproteogenic amino acids. In other embodiments, the peptidic oligomer comprises one or more proteogenic amino acids. In some embodiments when the peptidic oligomer comprises a proteogenic amino acid, the majority of amino acids in the peptidic oligomer are nonproteogenic amino acids.

The nonproteogenic amino acids comprises optionally substituted methylene groups CR₂ having a formula of HN(R³)(CRⁱ¹CRⁱ²)ₙC(═O)OH, wherein n is an integer greater than equal to 2, i is an index from 1 to n where 1 is a carbon adjacent to the terminal carboxyl group and n is the carbon adjacent the terminal amine group, and R³, Rⁱ¹, and Rⁱ¹ may be independently selected from H, alkyl, alkenyl, alkynyl, aryl, heteroaryl, amino, hydroxyl, carboxyl, amido, thiol, halo, alkylaryl, alkylheteroaryl alkylamino, alkylhydroxyl, alkylcarboxyl, alkylamido, alkylthiol, alkylhalo, optionally where R³ and either Rⁱ¹ or Rⁱ² may together form a heterocycle or optionally where Ru Rⁱ² and either R^{j1} or Rⁱ² may form a carbocyle.

The nonproteogenic amino acid may be a β-amino acid (i.e., n=2), a γ-amino acid (i.e., n=3), Δ-amino acid (i.e., n=4), a higher order amino acid (i.e., n is greater than equal to 5), or any combination thereof. In some embodiments, the peptidic oligomer comprises selected only from β-amino acids. In some embodiments, the peptidic oligomer comprises selected only from γ-amino acids. In some embodiments, the peptidic oligomer comprises selected only from Δ-amino acids. In some embodiments, the peptidic oligomer comprises selected only from higher order amino acids.

There are three general types of open-chain β-amino acids, depending on whether the substitution takes place at the carbon bearing the carboxyl group (α-position), the carbon bearing the amino group (β-position), or at both positions (α,β-disubstitution). In addition, cyclic p-amino acids may present the amino acid and the carboxylic groups as substituents of a carbocyclic ring or may incorporate the amino group in a heterocyclic ring. The nomenclature β²-and β³-amino acid may be used to indicate the position of the side chains, in order to distinguish positional isomers. The preceding may also be extended to γ-, Δ-, or higher order amino acids. Exemplary 0- and γ-amino acids are described, for example, in “Chapter 1: Structural Types of Relevant 0-Amino Acid Targets” in Juaristi, Eusebio and Vadim A Soloshonok. Enantioselective Synthesis of -Amino Acids John Wiley & Sons, Inc, 2005, 1-18 and Seeback et al. “The preceding may also be extended to γ-, Δ-, or higher order amino acids” Chemistry & Biodiversity 1:1111-1239 (2004)

Peptidic oligomer may be substituted with one or more substituents to further enhance solubility, enable cellular targeting, and/or enhance the stability of the biocompatible peptidic oligomer encapsulating the agent. Suitably, substitutions described above may be utilized with a targeting moieties, steric group, saccharide moiety, label, or any combination thereof.

In some embodiments, the peptidic oligomer is substituted with one or more targeting moieties. A targeting moiety may include one or more receptor ligands (e.g., a folate receptor ligand), binding partners, or antibodies, and the like.

In some embodiments, the peptidic oligomer is substituted with one or more steric groups, including tert-butyloxycarbonyl (BOC), 9-Fluorenylmethyloxycarbonyl (Fmoc), Carbobenzyloxy (Cbz), p-Methoxybenzyl carbonyl (Moz or MeOZ), acetyl (Ac), benzoyl (Bn), carbamate, p-methoxybenzyl (PMB), 3,4-dimethoxybenzyl (DMPM), p-methoxyphenyl (PMP), tosyl (Ts), and trichloroethyl chloroformate (troc).

In some embodiments, the peptidic oligomer may be substituted with one or more saccharide moieties, including monosaccharides, disaccharides, oligosaccharides, polysaccharides, glycans, aldoses, ketoses, amino sugars, intramolecular anhydridesfucose, and N-acetylglucoseamine.

In some embodiments, the nonproteogenic amino acids comprising a linear chain may be substituted with one or more labels. In some embodiments the labels may be used for detection, tracking, tracing, imaging, or other characterization while in other embodiments the labels are used for capturing. In some embodiments the label may be a fluorophore, phosphor, a magnetic bead, biotin, streptavidin, or an alkyne.

In some embodiments the amino acids are substituted with a targeting moiety, steric group, saccharide moiety, or label before assembling into a linear chain. In other embodiments, the nonproteogenic amino acids are assembled into a linear chain before substituting with a targeting moiety, steric group, saccharide moiety, or label.

In some embodiments, a folate receptor ligand, steric group, saccharide moiety, or label is conjugated to the nonproteogenic amino acid by a conjugation chemistry. In some embodiments the conjugation occurs through an amine, hydroxyl, carboxyl, thiol, or alkyne. Exemplary bioconjugation chemistries, including without limitation, ‘Click’ chemistries, such as (Copper (I)-catalyzed azide-alkyne cycloaddition (CuAAC), strain-promoted azide-alkyne cycloaddition (SPAAC), or strain-promoted alkyne-nitrone cycloaddition (SPANC); amine conjugation with NHS ester, isocyanate, isothiocyanate, or an anhydride; thiol conjugation with a maleimide or disulfide; carboxylic acid conjugation with a carbodiimide coupling; or other suitably conjugation chemistry.

The peptidic oligomers described herein may be biocompatible. Biocompatible refers to peptidic oligomers that demonstrate no statistically significant activity in a cellular environment at a particular amount. When administered to a subject, biocompatible my refer to peptidic oligomers that demonstrate no statistically significant activity to non-cancerous cells or tissue but may demonstrate activity against cancerous cells, neoplasms, or tumors. In some embodiments, statistically significant activity may be determined by a cellular toxicity assay, a metabolic assay, a protein expression assay, a protein activity assay, or any other suitable assay for investigating a cellular system. In particular embodiments, biocompatible refers to a peptidic oligomer that is not statistically toxic to a particular cell at a given particular amount.

An agent can be defined as any compound that maybe delivered or increased in concentration. In some embodiments, aspects of desirable agents include compounds that would benefit from increased solubility, increased efficacy, or increase bioavailability.

In some embodiments, the agent is an active pharmaceutical ingredient (API). As used herein, API refers to any substance or combination of substances used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions.

The peptidic oligomer may be a sensitizer. As used herein, a sensitizer or sensitizing agent is any compound or composition that improves or increases the activity, decreases resistance to, or improves a therapeutic outcome of another compound or composition when used in combination with the sensitizer.

In some embodiments, the agent is a diagnostic agent or probe. As used herein, diagnostic agent refers to any substance or combination of substances used to examine a subject in order to detect impairment of normal functions. As used herein, a probe refers to any substance or combination of substances used to examine a chemical environment, including in vivo, ex vivo, and in vitro chemical environments. Suitably diagnostic agents or probes may include substances having radiolabels, fluorescence labels, bioluminescence labels, and the like.

In some embodiments, the agent is an agrochemical. As used herein, agrochemical refers to any substance or combination of substances used in agriculture. Exemplary agrochemicals include, without limitation, herbicides, pesticides, safeners, hormones, biostimulants, and the like.

The agent and the biocompatible peptidic oligomer form a complex with the agent encapsulated within the peptidic oligomer. In some embodiments, the oligomer encapsulates the agent in a 1:1 ratio.

The agent may be a linear compound. As used herein, a linear compound is one that does not have any cyclic or aryl structures. Linear compounds may have one or more branches comprised of a non-hydrogenic atom such as C, N, O, S, P, or halo. To accommodate the agent within the encapsulating peptidic oligomer, a branch may have 1-8, 1-6, 1-4, or 1-3 non-hydrogenic atoms. Exemplary branches, include without limitation, are alkyl, alkenyl, or alkynyl chains (e.g., methyl, ethyl, propyl, or butyl) which may be optionally substituted with one or more of a amine, oxo, carboxyl, hydroxyl, amido, or any combination thereof, amines (e.g., NH₂), oxo, carboxyl, hydroxyl, or amido.

Exemplary linear agents include, without limitation, the anti-cancer agent, sulforaphane, as well as dimercaprol, bethanechol, mechlorethamine, methimazole, disulfiram, acetazolamide, busulfan, meprobamate, thiotepa, echothiophate, acetylcysteine, aminocaproic acid, pregabalin, acetylcholine, hydroxyurea, ethambutol, carbachol, carmustine, isoflrane, valproic acid, levocarnitine, methacholine, ethanolamine oleate, penicillamine, amifostine, or alendronate.

In some embodiments the agent has a molecular weight no higher than 350 grams/mole. In some embodiments the agent is an active pharmaceutical ingredient with a molecular weight no higher than 350 grams/mole.

As previously discussed, the disclosed peptidic oligomers may encapsulate an agent in favorable conditions for encapsulation. In one embodiment, the favorable conditions for encapsulation may include dissolving the peptidic oligomer in water by sonication for a suitably duration. In other embodiments an aqueous buffer may be used. To this aqueous solution the agent may be added at a desired amount, and vortexed. This solution may remain covered at room temperature. In further embodiments, this solution is centrifuged to precipitate excess peptide and collect the supernatant.

The peptidic oligomers and peptidic oligomers encapsulating an agent disclosed herein may be formulated as pharmaceutical compositions. The pharmaceutical compositions may include an effective amount of the peptidic oligomer or encapsulated complex and one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 500 mg/kg body weight (preferably about 0.5 to 100 mg/kg body weight, more preferably about 0.1 to 75 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 mM.

In some embodiments the pharmaceutical composition is administered in an aqueous solution. In other embodiments, the pharmaceutical composition is administered in an aqueous buffer solution, including phosphate buffer solution (PBS), 2-[bis(2-hydroxyethyl)amino]-2-(hydroxymethyl)propane-1,3-diol (Bis-tris), 2-[4-(2-Hydroxyethyl)-1-piperazine]ethanesulfonic acid (HEPES), 3-Cyclohexylamino-1-propanesulfonic acid (CAPS), 1, 4-Piperazinebis(ethanesulfonic acid) (PIPES), N-(2-Acetamido)iminodiacetic acid, N-(Carbamoylmethyl)iminodiacetic acid (ADA), N, N-Bis(2-hydroxyethyl)glycine, Diethylolglycine (Bicine), 2-[Tris(hydroxymethyl)methylamino]-1-ethanesulfonic acid (TES), sodium; 2-morpholin-4-ylethanesulfonate (MES), and other common aqueous buffer solutions well known in the art.

As indicated above, pharmaceutically acceptable salts of the peptidic oligomer or complex of the peptidic oligomer encapsulating an agent are contemplated and also may be utilized in the disclosed methods. The term “pharmaceutically acceptable salt” as used herein, refers to salts of the peptidic oligomers encapsulating an agent which are substantially non-toxic to living organisms. Typical pharmaceutically acceptable salts include those salts prepared by reaction of the peptidic oligomers encapsulating an agent as disclosed herein with a pharmaceutically acceptable mineral or organic acid or an organic or inorganic base. Such salts are known as acid addition and base addition salts. It will be appreciated by the skilled reader that most or all of the peptidic oligomers encapsulating an agent as disclosed herein are capable of forming salts and that the salt forms of pharmaceuticals are commonly used, often because they are more readily crystallized and purified than are the free acids or bases.

The peptidic oligomer or complex utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The peptidic oligomer or complex utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The peptidic oligomer or complex utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The peptidic oligomer or complex utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The peptidic oligomer or complex utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the peptidic oligomer or complex may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The peptidic oligomer or complex employed in the compositions and methods disclosed herein may be administered as pharmaceutical compositions and, therefore, pharmaceutical compositions incorporating the peptidic oligomers or complexes are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed complex, which effective amount is related to the daily dose of the complex to be administered. Each dosage unit may contain the daily dose of a given complex or each dosage unit may contain a fraction of the daily dose, such as one-half or one-third of the dose. The amount of each peptidic oligomer or complex to be contained in each dosage unit can depend, in part, on the identity of the particular complex chosen for the therapy and other factors, such as the indication for which it is given. The pharmaceutical compositions disclosed herein may be formulated so as to provide quick, sustained, or delayed release of the active ingredient after administration to the patient by employing well known procedures. The peptidic oligomers or complexes for use according to the methods of disclosed herein may be administered as a peptidic oligomer or single complex or a combination of peptidic oligomers or complexes.

Pharmaceutically acceptable esters and amides of the complexes can also be employed in the compositions and methods disclosed herein.

In addition, the methods disclosed herein may be practiced using solvate forms of the complexes or salts, esters, and/or amides, thereof. Solvate forms may include ethanol solvates, hydrates, and the like.

Methods for treating subjects with the peptidic oligomers or complexes disclosed herein are provided. Suitably the method for treating a subject comprises administering to the subject an effective amount of one or more peptidic oligomers or one or more complexes disclosed herein or a pharmaceutical composition comprising the effective amount of one or more of the complexes disclosed herein. As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with one or more of the complexes, agents, or peptidic oligomers disclosed herein. In some embodiments, the subject is responsive to therapy with one or more of the complexes disclosed herein in combination with one or more additional therapeutic agents. In some embodiments, the subject is responsive to therapy with one or more of the complexes disclosed herein in combination with one or more peptidic agents. For example, a “subject in need of treatment” may include a subject in need of treatment for a cancer. In some embodiments, the subject in is need of treatment for a breast cancer.

In some embodiments, the active pharmaceutical ingredient is an antitumor agent, an antioxidant, an anti-inflammatory agent, an antimicrobial agent, or a neuroprotectant. An active pharmaceutical ingredient used in some embodiment is sulforaphane.

Another aspect of the technology provides for a method for treating a subject in need of a treatment for cancer comprising administering a pharmaceutical composition comprising a peptidic oligomer and a pharmaceutically acceptable carrier, excipient, or diluent to the subject. In some embodiments, the peptidic oligomer encapsulates an antitumor agent. As demonstrated in the Examples the peptidic oligomer may be an antitumor agent capable of inhibiting the growth or proliferation of cancerous cells or tumors.

As also demonstrated in the Examples, the peptidic oligomer may be sensitize cancerous cells or tumors to an encapsulated antitumor agent, thereby inhibiting the growth or proliferation of cancerous cells or tumors to a greater extent then if the antitumor agent was administer without the peptidic oligomer.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In some embodiments, the methods described herein are practiced in vivo. In other embodiments, the methods described herein are practiced in vitro or ex vivo.

As used herein the term “effective amount” refers to the amount or dose of the peptidic oligomer or complex that provides the desired effect. In some embodiments, the effective amount is the amount or dose of the peptidic oligomer or complex, upon single or multiple dose administration to the subject, which provides the desired effect in the subject under diagnosis or treatment. Suitably the desired effect may be inhibiting the growth or proliferation of or killing the cancerous cells (e.g., breast cancer cells) or tumors in the subject or reversing the progression or severity of resultant symptoms associated with the cancerous cells or tumors.

An effective amount can be readily determined by those of skill in the art, including an attending diagnostician, by the use of known techniques and by observing results obtained under analogous circumstances. In determining the effective amount or dose of complex administered, a number of factors can be considered by the attending diagnostician, such as: the species of the subject; its size, age, and general health; the degree of involvement or the severity of the disease or disorder involved; the response of the individual subject; the particular complex administered; the mode of administration; the bioavailability characteristics of the preparation administered; the dose regimen selected; the use of concomitant medication; and other relevant circumstances.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Synthesis of Alpha, Beta, and Gamma Peptidic Oligomers

Solid phase peptide synthesis was used to synthesize α-, β-, and γ-peptidic oligomers using FMOC chemistry and 2-chlorotrityl resin. Methods were obtained from https://www.peptide.com/resources/.

The hexamer polypeptide of beta, referred to as B6, was synthesized from Boc-3-(Fmoc-amino)-L-alanine, Nα-Boc-Nβ-Fmoc-L-2,3-diaminopropionic acid (CAS #122235-70-5). The hexamer polypeptide of gamma, referred to as G6, was synthesized from Nα-Boc-Nγ-Fmoc-L-2,4-diaminobutyric acid (CAS #117106-21-5).

All FMOC groups were removed from the peptide before cleavage from the resin using standard methods. Peptides were cleaved from resin using 30% Hexafluoroisopropanol (CAS #920-66-1) in dichloromethane (CAS #75-09-2) according to the procedure described in Hickey, Jennifer L., J. Med. Chem. 2016, 59, 11, 5368-5376. All BOC groups were left attached to the peptide.

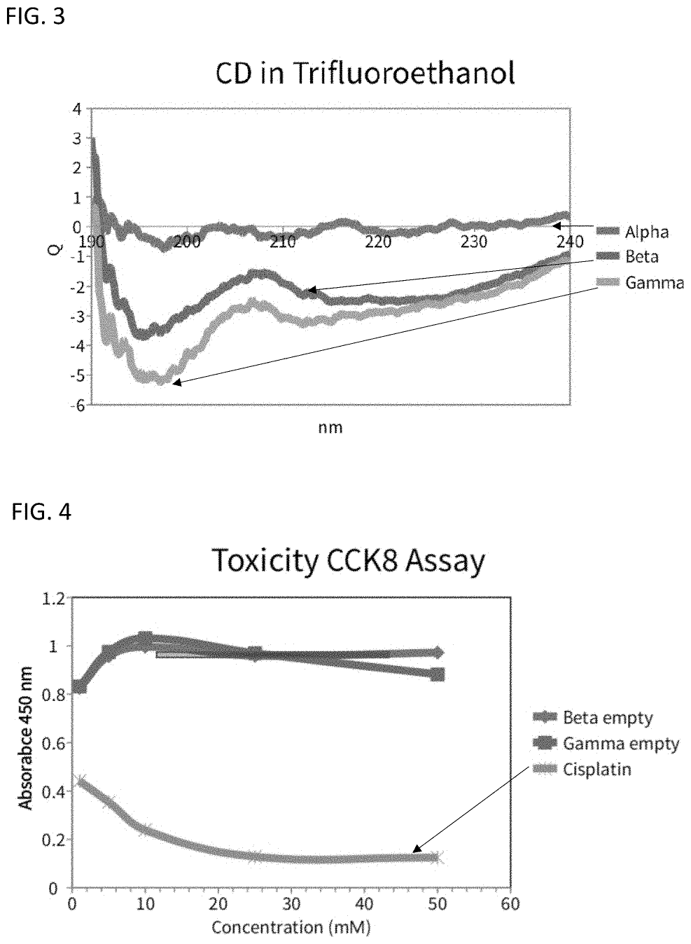

Characterization by circular dichroism (FIG. 3) shows peaks at ˜193, 208, and 222 nm which indicate the ability to form alpha helices.

Biocompatibility of the peptidic oligomers was characterized in a toxicity assay of the empty beta and gamma chains performed with mouse 4T1 breast cancer cells (FIG. 4). Cisplatin was used as a toxicity reference. Cells were incubated with varying concentrations for 48 hours, followed by CCK8 reagent. The absorbance at 450 nm was recorded. This data indicates that as the concentration of cisplatin increases, the number of viable cells decreases. As predicted, empty beta and gamma alpha helices show biocompatibility in that they do not display cellular toxicity.

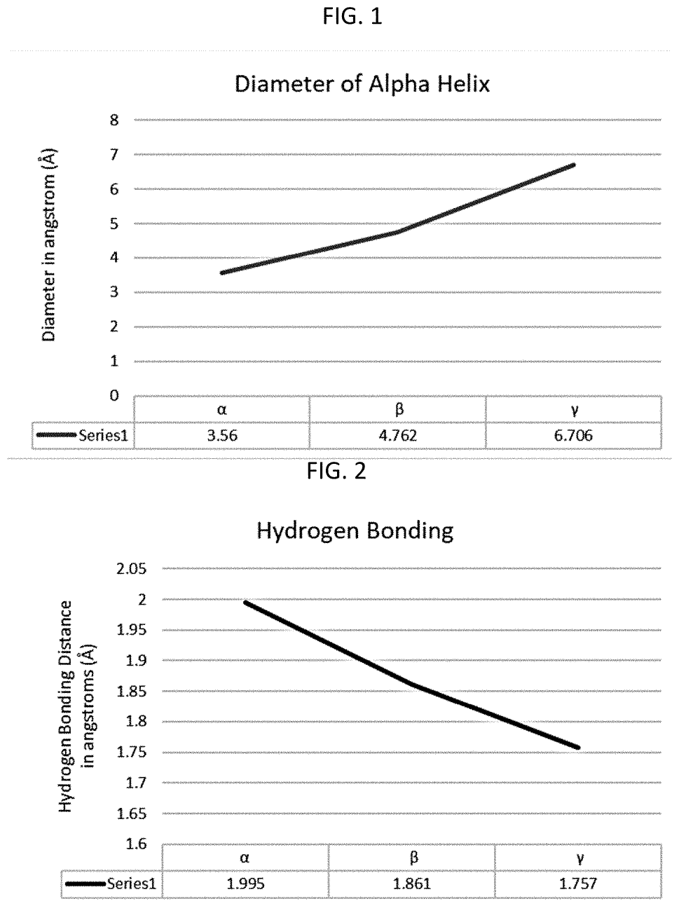

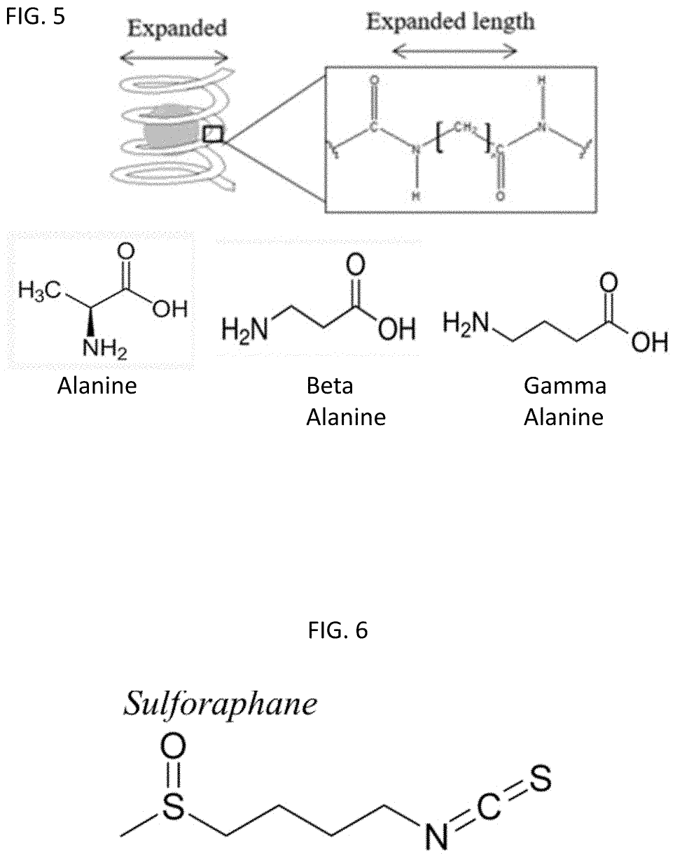

The combined teachings of FIGS. 1-2 and 5 illustrate an expanded α-helix system and the skeletal structures of alanine, beta, and gamma and the change in size of the helical diameter of alpha, beta, and gamma alanine peptidic oligomers.

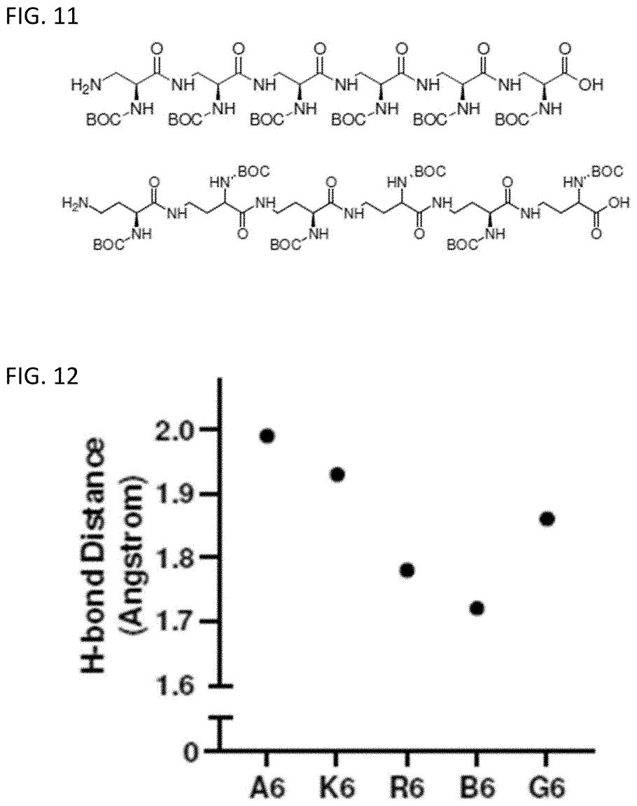

Sequences of β and γ 2-8 amino acids long were synthesized and screened qualitatively using the fluorescence and functional assays described earlier (FIG. 11).

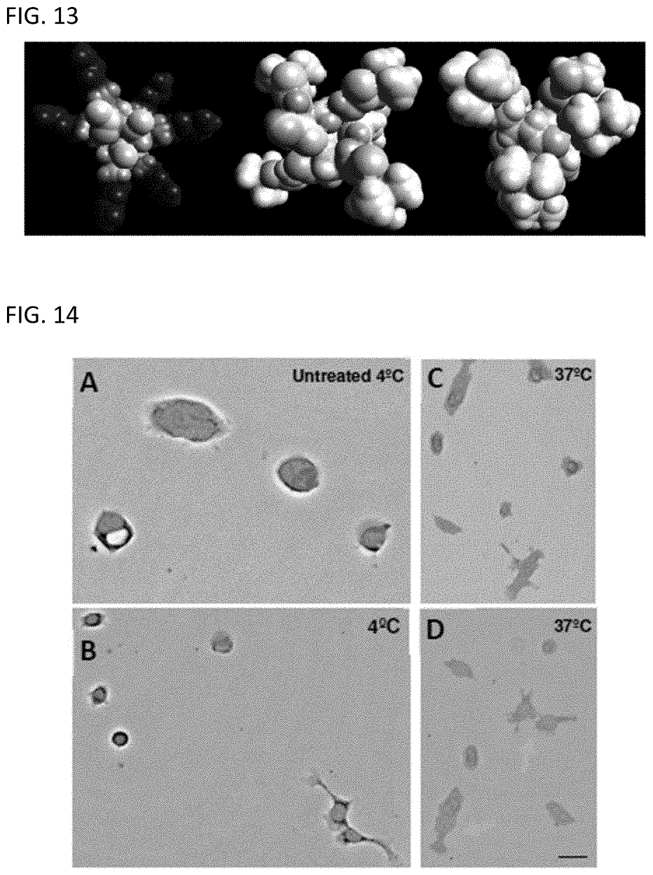

FIG. 12 shows that H-bond distance decreases across hexa alanine (A6), hexa lysine (K6), and hexa arginine (R6). The H-bond distance for B6 is even smaller than R6, implying that it is more stable. The distance of G6, is between K6 and R6, but still shorter than A6. Since B6 and G6 are β and γ amino acids respectively, the extra aliphatic carbon atoms in the backbone provide more steric flexibility and “floppiness” that could facilitate orientations favorable for H-bond formation, and therefore more stable alpha helices. CPPs cationic side chains facilitate binding to the negatively charged parts of the cell membrane. The Boc groups on our engineered CPPs are not polar, which makes them chemically less active. Electron density diagrams calculated in Avogadro show R6 is indeed cationic, with B6 and G6 having essentially neutral side chains, but still having a dipole with positive and negative poles corresponding with the amine and carbonyl terminal ends respectively, as seen in FIG. 13.

After structural characterization B6 and G6, the peptidic oligomers were tagged with FITC and incubated with 4T1 cells. FIG. 14 shows room temperature in vitro cell assays performed. Fluorescence is found evenly distributed on the cell at room temperature, implying both energy dependent and independent penetration mechanisms are possible. The same assay was performed at 4° C. and no fluorescence is observed, thus the primary mechanism of penetration is the energy-dependent endocytosis mechanisms, and not energy-independent direct binding translocation.

### Example 2: Encapsulation of Sulforaphane

D, L sulforaphane (CAS #4478-93-7) (FIG. 6) was encapsulated by the biocompatible peptidic oligomers, B6 and G6.

A saturated solution of the peptidic oligomer was dissolved in water by sonication for 10 minutes. Sulforaphane was added to achieve a peptide:sulforaphane ratio of 1:0-1.25 mole:mole ratio. The solution was mixed by vortexing. The solution was left covered at room temperature overnight. Finally, the solution was centrifuged to precipitate excess peptidic oligomer and the clear supernatant was collected.

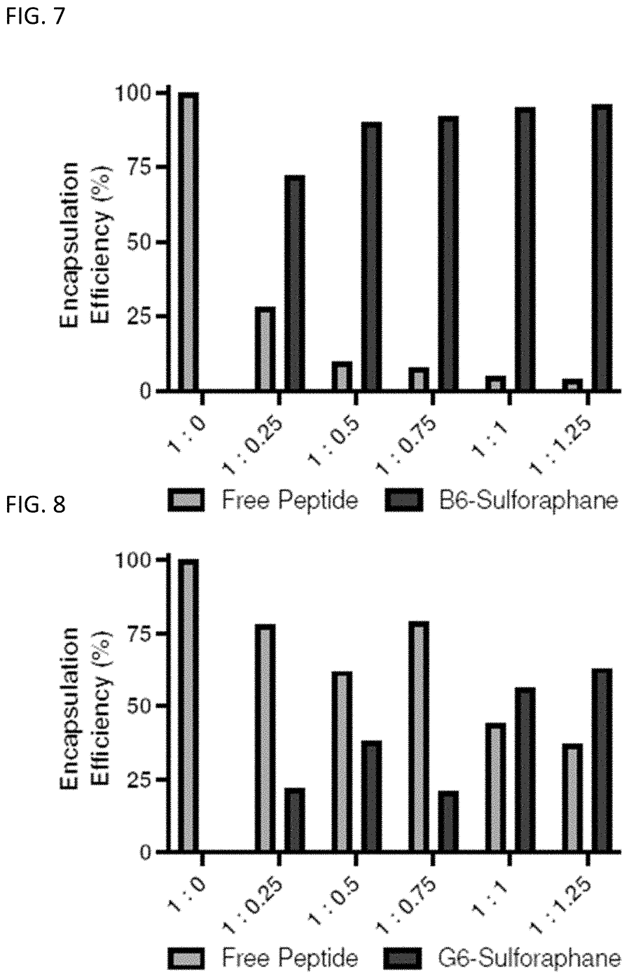

The unprocessed solutions were analyzed by liquid chromatography-mass spectrometry (LCMS) to determine encapsulation efficiency (EE) as presented in FIGS. 7-8. B6 shows 90% encapsulation efficiency at 1:0.5 molar ratio. G6 has a maximum EE of 60% at 1:1.25 molar ratio. The area under peaks from LC corresponding to each mass were compared to determine EE where the respective m/z peaks are B6 (1135 g/mol), BS (1312 g/mol), G6 (1219 g/mol) and GS (1396 g/mol).

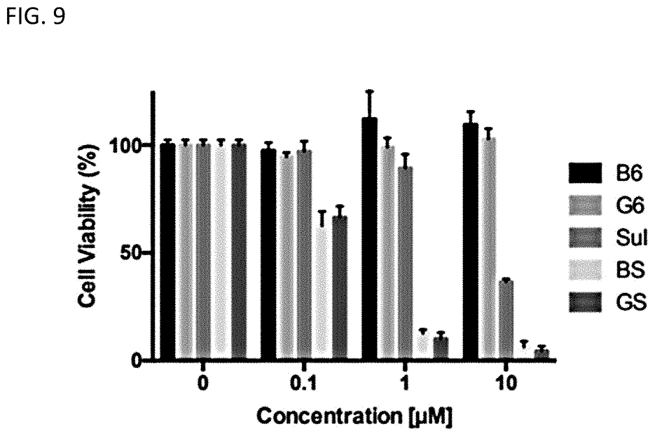

The encapsulated sulforaphane was characterized in an in vitro assay for cell viability, shown in FIG. 9. A range of concentrations of B6, G6, free sulforaphane, sulforaphane encapsulated by B6 (called “BS”), and sulforaphane encapsulated by G6 (called “GS”) were tested for biological activity against 4T1 breast cancer cells. The assay was run for 72 hours with the CellTiter-Glo assay to determine cell viability. Results show that the free peptides B6 and G6 are biocompatible and do not affect cell viability. Free sulforaphane shows significant activity at 10 mM. The encapsulated constructs, BS and GS, show significant activity at as low as 0.1 mM, 100× less than the free sulforaphane. Sulforaphane itself alone showed significant activity with an ED50 of 6 mM, thus it is able to penetrate the cell membrane and be metabolized. B6S and G6S show significant activity at lower concentration with an ED50 of 0.3 mM, an effect greater than sulforaphane alone.

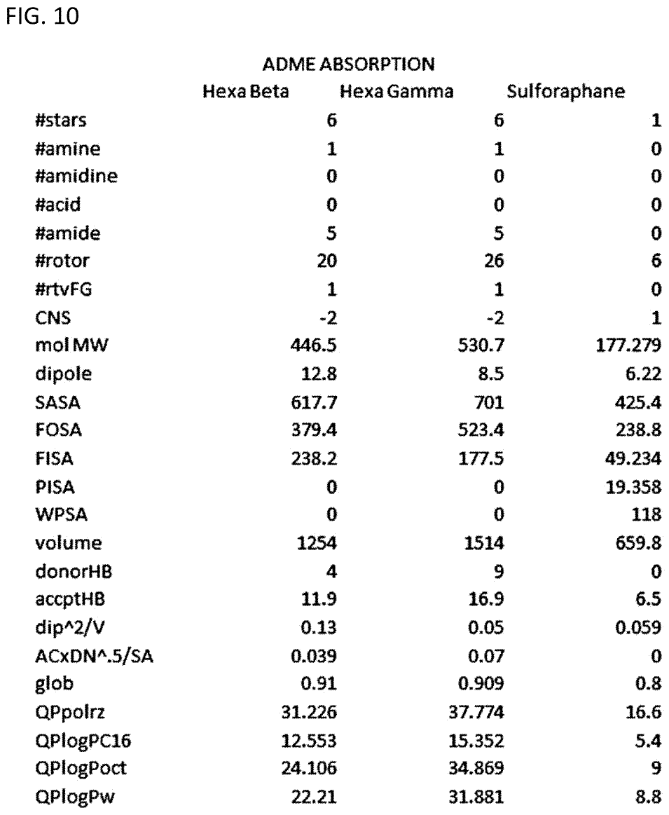

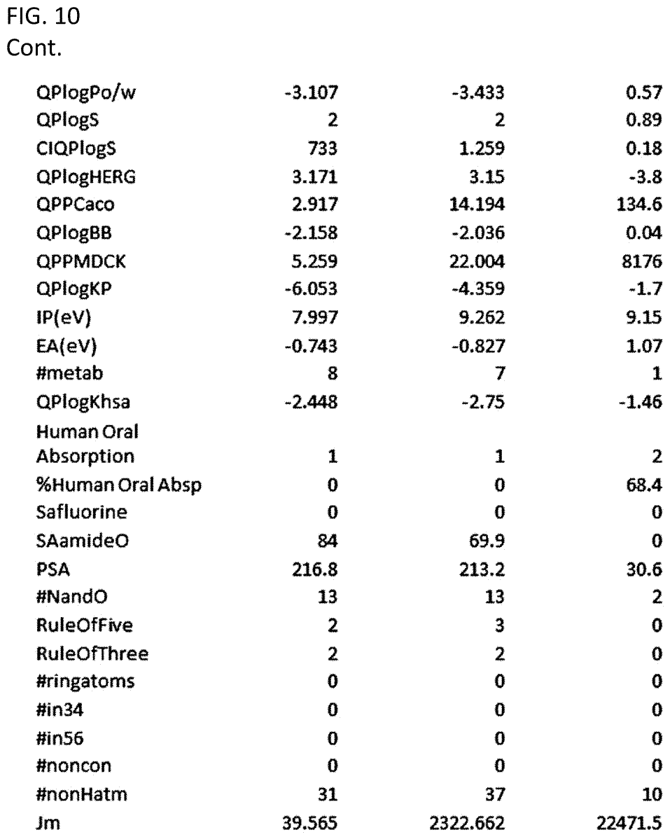

FIG. 10 shows a table of pharmaceutical absorption (Absorption, distribution, metabolism, and excretion; ADME) rates. Using Schrodinger, Qikprop acts as a rapid ADME prediction of drug candidates. Hexa alanine, Beta, Gamma, and sulforaphane were analyzed for relevant pharmaceutically relevant properties. Alanine exhibits properties that fall outside of normal range predictions to perform well in clinical testing. Hexa Beta, and Gamma shows greater probability of success (Qppolrz) in living organisms than sulforaphane. Sulforaphane indicated the least biocompatibility within kidney (QPPMDCk), binding to albumin (QPlogKhsa), and a weak polar component (WPSA), as expected for an oil.

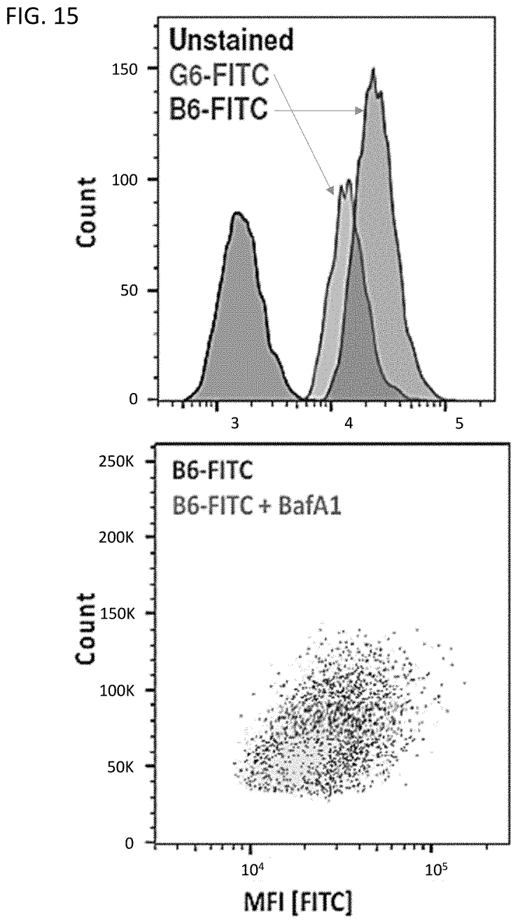

Bafilomycin A1 (BafA1; 328120001; ThermoFisher Scientific), a vacuolar ATPase inhibitor, blocked FITC-labeled CPP (FIG. 15). Vacuolar ATPase aids in the transport to the interior of acidic organelles and BafA1 efficiently neutralizes the luminal pH, inhibits acidic hydrolases, and impairs the fusion among acidic organelles. Thus, these results also show that B6 and G6 are not taken up by the cell by direct translocation but that this occurs via an energy-dependent endocytic pathway.

### Example 3: In Vivo Efficacy

B6Sulforophane was dissolved in sterile H₂O and injected intraperitoneal (IP.) in 8-weeks old female mice. Two (2) received 1 mg/ml in a total volume of 150 ul, and 2 received 2 mg/kg in 300 ul volume. The mice were observed for 15 minutes and no signs of acute toxicity or discomfort (e.g. writhing, stretching and back arching, decrease activity, abnormal gait), were noted. The mice were observed again at 2 hr and 24 hr after injection and no signs of gross toxicity were noted (e.g. loss in body weights, change in coat, change in behavior).

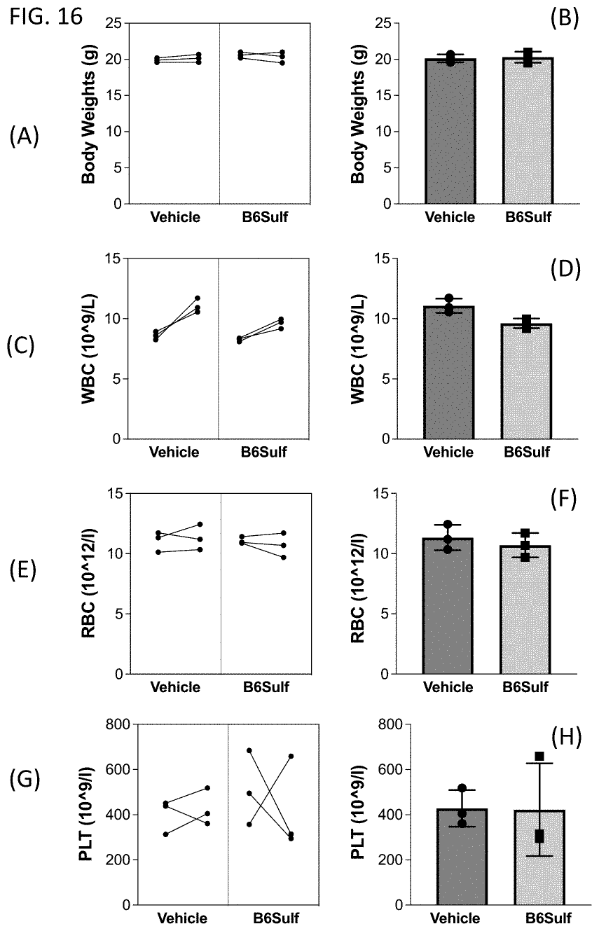

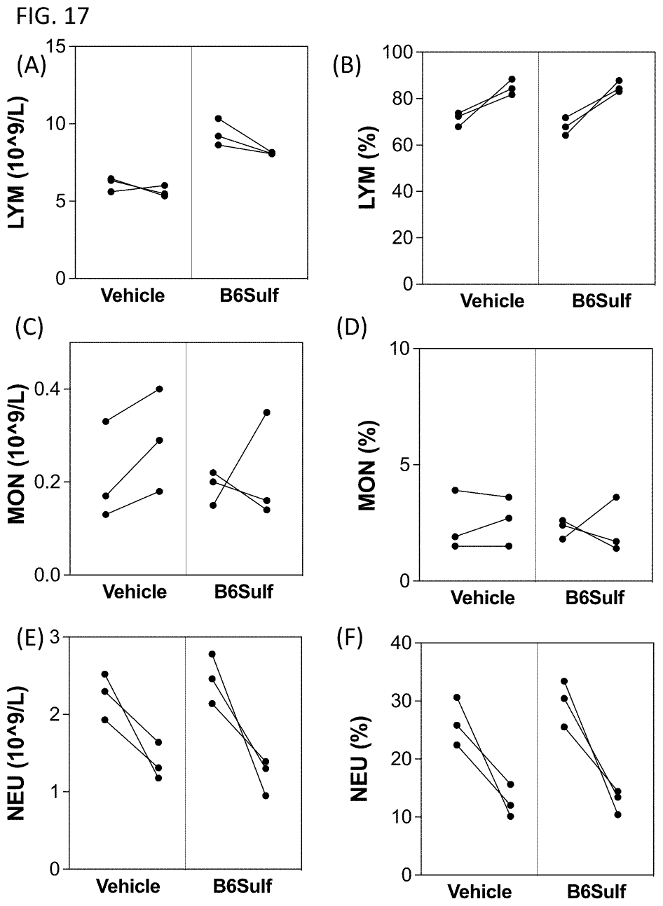

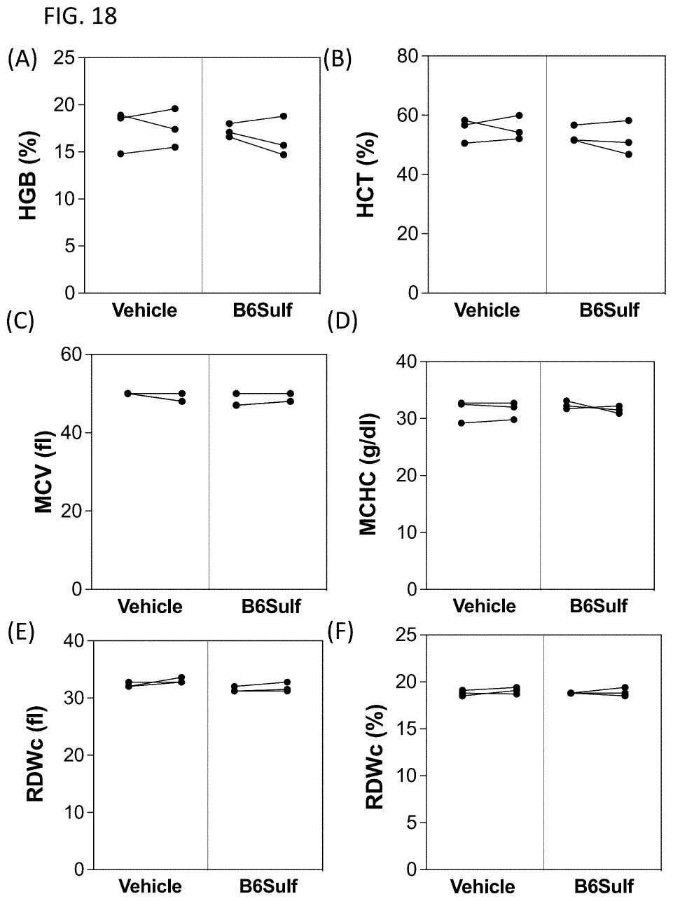

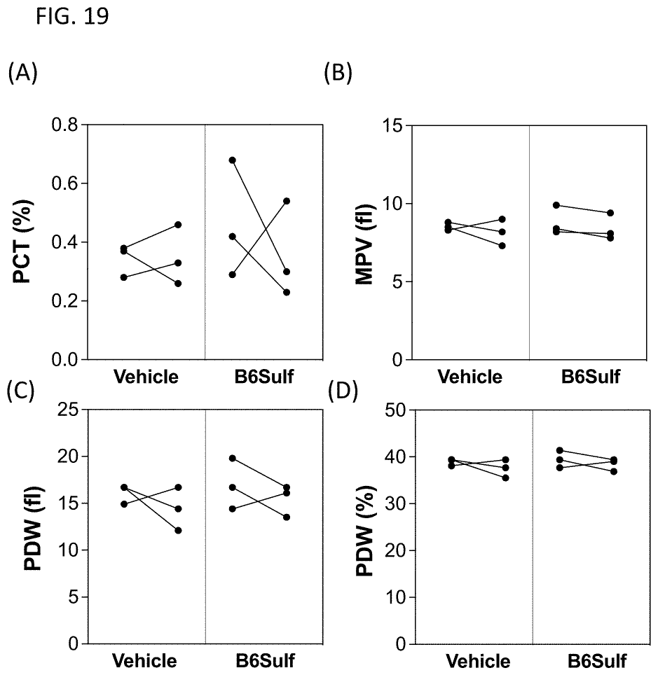

For complete blood count (CBC) assessment blood was obtained via the tail veins 2 and 24 hr after 2 mg/kg B6Sulforophane or vehicle and analyzed on a 3-part differential hematology analyzer (Vetscan HM5). As shown in FIG. 16, B6Sulf does not induce acute or long-term toxicity. FIG. 17, B6Sulf does not induce acute or long-term leukocyte toxicity. FIG. 18 shows that B6Sulf does not induce acute or long-term erythrocyte toxicity and FIG. 19 shows B6Sulf does not induce acute or long-term thrombocyte toxicity.

Triple-negative 4T1 breast cancer cells (#CRL-2539) were purchased from American Type Culture Collection (ATCC) and cultured in DMEM supplemented with 10% fetal bovine serum (FBS) and 1% penicillin+streptomycin (P/S) and passaged biweekly. Female Balb/c mice 6-weeks of age (Jackson Labs, ME) were allowed to acclimatize for 7 days and subsequently inoculated subcutaneously in the rear limb with 1×10⁵ 4T1 cells. After 5 days, tumors had grown to an average size of 7 mm in diameter (˜150 mm³) at which time the animals were randomly assigned treatment groups. Namely, Vehicle (H₂O), B6, sulforaphane, or 2 mg/kg B6Sulforophane (n=4-8 per group). The vehicle and compounds were injected intraperitoneal in 150 ul volumes daily (q1dx10). B6 and sulforaphane were injected in the molar equivalent doses as the quantities found in B6S. Experiments were approved by the University of Arkansas for Medical Sciences Institutional Animal Care and Use Committee and performed in accordance with relevant regulations and guidelines. Body weights were recorded daily and at the termination of the study tumors, spleens, small intestines, ceca and colons were harvested and measured 24 h after the last injections.

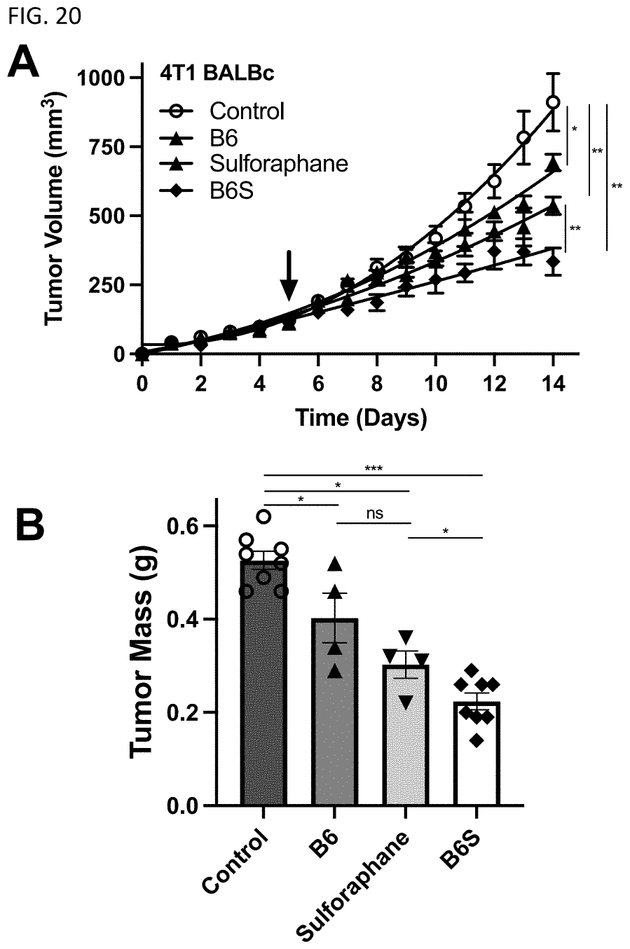

FIG. 20 shows that B6Sulforophane inhibits triple-negative 4T1 breast carcinoma to a greater extent than either free sulforophane or B6 alone. Thus the peptidic oligomer sensitizes the subject to the API, sulforophane. Surprisingly, B6 alone demonstrated statistically significant in vivo activity against the tumor. In contrast, in vitro testing demonstrated little to no activity for the peptidic oligomers (FIG. 9) at all concentrations tested.

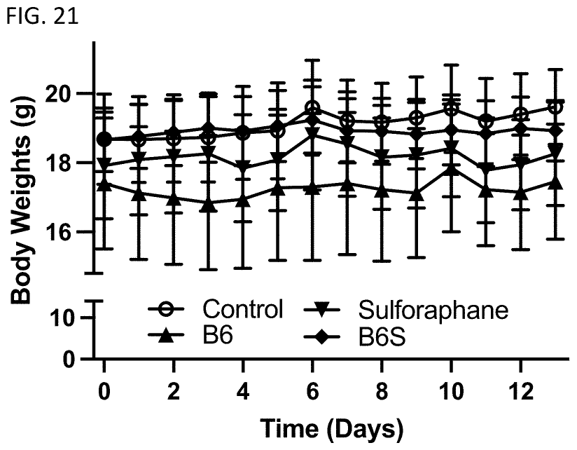

FIG. 21 shows B6Sulforophane treatment does not induce general toxicity in 4T1-bearing mice as monitored by body weight.

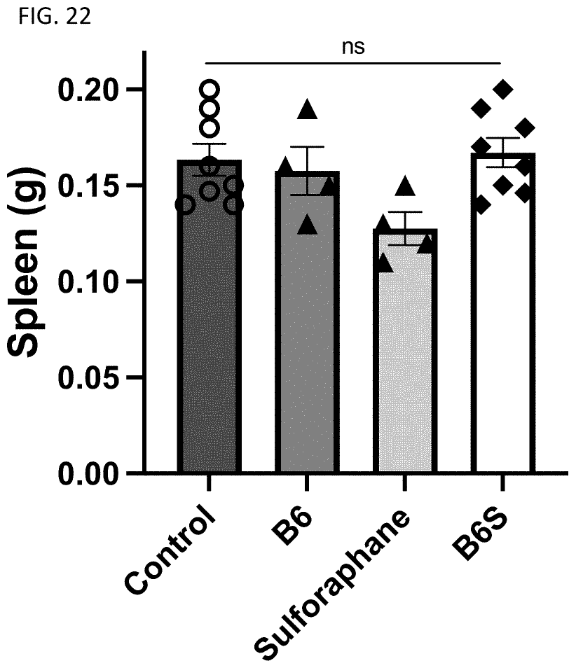

FIG. 22 shows B6Sulforophane treatment does not induce general splenocytes toxicity in 4T1-bearing mice.

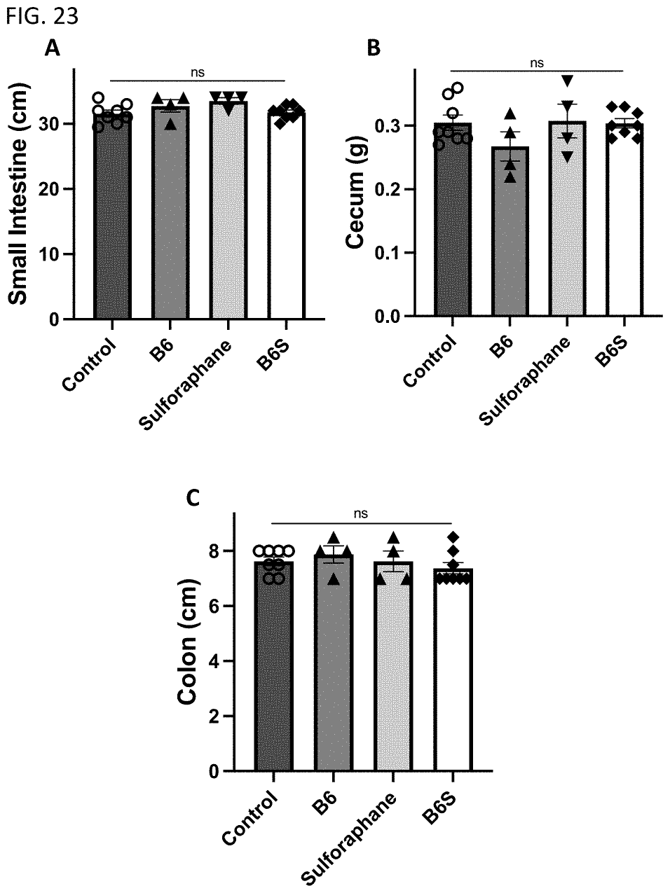

FIG. 23 shows B6Sulforophane treatment does not induce general gastrointestinal tract toxicity in 4T1-bearing mice.

What is claimed is:

1. A complex comprising a peptidic oligomer encapsulating an agent, or a pharmaceutically acceptable salt thereof, wherein the peptidic oligomer comprises a linear chain formed from a multiplicity of independently selected nonproteogenic amino acids joined by amide bonds and the nonproteogenic amino acids comprise two or more optionally substituted methylene groups interposed between a terminal amino and a terminal carboxylic acid or ester group.

2. The complex of claim 1, wherein 1 peptidic oligomer encapsulates 1 agent.

3. The complex of claim 1, wherein the agent is an active pharmaceutical ingredient, a diagnostic agent, a probe, or an agrochemical.

4. The complex of claim 3, wherein the agent is the active pharmaceutical ingredient.

5. The complex of claim 4, wherein the active pharmaceutical ingredient is sulforaphane.

6. The complex of claim 1, wherein the agent comprises a linear chain optionally comprising one or more branches extending therefrom, wherein the linear chain comprises carbon and optionally one or more heteroatoms.

7. The complex of claim 1, wherein the nonproteogenic amino acids comprise optionally substituted β-amino acids or optionally substituted γ-amino acids.

8. The complex of claim 7, wherein the nonproteogenic amino acids comprise optionally substituted β-alanine.

9. The complex of claim 7, wherein the nonproteogenic amino acids comprise optionally substituted γ-alanine.

10. The complex of claim 1, wherein the peptidic oligomer is substituted with one or more targeting moieties, one or more steric groups, one or more saccharide moieties, one or more labels, or any combination thereof.

11. The complex of claim 1, wherein the peptidic oligomer comprises between 4-8 nonproteogenic amino acids.

12. The complex of claim 1, wherein the peptidic oligomer is biocompatible.

13. A pharmaceutical composition comprising an active pharmaceutical ingredient encapsulated by the peptidic oligomer according to claim 1 and a pharmaceutically acceptable carrier, excipient, or diluent.

14. The pharmaceutical composition of claim 13, wherein the molar ratio of peptidic oligomer to active pharmaceutical ingredient is 1:1.

15. The pharmaceutical composition of claim 13, wherein the active pharmaceutical ingredient comprises a linear chain optionally comprising one or more branches extending therefrom, wherein the linear chain comprises carbon and optionally one or more heteroatoms.

16. The pharmaceutical composition of claim 13, wherein the active pharmaceutical ingredient is sulforaphane.

17. A method for treating a subject comprising administering the pharmaceutical composition according to claim 13 to the subject, wherein the subject is in need of the active pharmaceutical ingredient.

18. The method of claim 17, wherein the subject is in need of a treatment for a cancer.

19. The method of claim 18, wherein the subject is in need of a treatment for a breast cancer.

20. The method of claim 17, wherein the active pharmaceutical ingredient is an antitumor agent, an antioxidant, an anti-inflammatory agent, an antimicrobial agent, or a neuroprotectant.

21. The method of claim 20, wherein the active pharmaceutical ingredient is sulforaphane.

22. A method for treating a subject in need of a treatment for cancer comprising administering a pharmaceutical composition comprising a peptidic oligomer and a pharmaceutically acceptable carrier, excipient, or diluent to the subject, the peptidic oligomer comprising a linear chain formed from a multiplicity of independently selected nonproteogenic amino acids joined by amide bonds and the nonproteogenic amino acids comprise two or more optionally substituted methylene groups interposed between a terminal amino and a terminal carboxylic acid or ester group.

23. The method of claim 22, wherein the cancer is a breast cancer.

24. The method for preparing the composition according to claim 1, the method comprising incubating the agent with the peptidic oligomer in a solvent under conditions sufficient for encapsulating the agent with the peptidic oligomer.
